(19) United States
(12) Patent Application Publication
(10) Pub. No.: US 2012/0059442 A1
(43) Pub. Date: Mar. 8, 2012

(54) STIMULATION LEAD, STIMULATION SYSTEM, AND METHOD FOR LIMITING MRI INDUCED CURRENT IN A STIMULATION LEAD

(76) Inventors: Timothy J. Cox, Leonard, TX (US); Ana P. Keef, Allen, TX (US)

(21) Appl. No.: 12/479,118

(22) Filed: Jun. 5, 2009

Related U.S. Application Data
(60) Provisional application No. 61/059,048, filed on Jun. 5, 2008.

(51) Int. Cl. A61N 1/05

(52) U.S. Cl. 607/116

(57) ABSTRACT

In one embodiment, a percutaneous stimulation lead for electrically stimulating tissue of a patient, comprises: a plurality of electrodes being electrically coupled to a plurality of terminals through a plurality of conductors within a lead body of the lead, wherein each electrode comprises a respective first surface exposed on an exterior surface of the stimulation lead to conduct current to or from tissue of the patient and a respective second surface disposed within an interior of the stimulation lead, the plurality of electrodes are arranged such that adjacent pairs of electrodes are capacitively coupled through a first surface of a first electrode of the respective pair and a respective second surface of a second electrode of the respective pair to substantially block current flow between adjacent electrodes at stimulation frequencies and to substantially pass current between adjacent electrodes at MRI frequencies.

### CROSS-REFERENCE TO RELATED APPLICATIONS

This application claims the benefit of U.S. Provisional Application No. 61/059,048, filed Jun. 5, 2008, which is incorporated herein by reference.

### TECHNICAL FIELD

The present application is generally related to limiting MRI induced current in a stimulation lead such as a neurostimulation lead, a cardiac stimulation lead, and/or the like.

### BACKGROUND

Neurostimulation systems are devices that generate electrical pulses and deliver the pulses to nerve tissue to treat a variety of disorders. Spinal cord stimulation (SCS) is an example of neurostimulation in which electrical pulses are delivered to nerve tissue in the spine for the purpose of chronic pain control. Other examples include deep brain stimulation, cortical stimulation, cochlear nerve stimulation, peripheral nerve stimulation, vagal nerve stimulation, sacral nerve stimulation, etc. While a precise understanding of the interaction between the applied electrical energy and the nervous tissue is not fully appreciated, it is known that application of an electrical field to spinal nervous tissue can effectively mask certain types of pain transmitted from regions of the body associated with the stimulated nerve tissue. Specifically, applying electrical energy to the spinal cord associated with regions of the body afflicted with chronic pain can induce “paresthesia” (a subjective sensation of numbness or tingling) in the afflicted bodily regions. Thereby, paresthesia can effectively mask the transmission of non-acute pain sensations to the brain.

Neurostimulation systems generally include a pulse generator and one or several leads. The pulse generator is typically implemented using a metallic housing that encloses circuitry for generating the electrical pulses. The pulse generator is usually implanted within a subcutaneous pocket created under the skin by a physician. The leads are used to conduct the electrical pulses from the implant site of the pulse generator to the targeted nerve tissue. The leads typically include a lead body of an insulative polymer material with embedded wire conductors extending through the lead body. Electrodes on a distal end of the lead body are coupled to the conductors to deliver the electrical pulses to the nerve tissue

There are concerns related to the compatibility of neurostimulation systems with magnetic resonance imaging (MRI). MRI generates cross-sectional images of the human body by using nuclear magnetic resonance (NMR). The MRI process begins with positioning the patient in a strong, uniform magnetic field. The uniform magnetic field polarizes the nuclear magnetic moments of atomic nuclei by forcing their spins into one of two possible orientations. Then an appropriately polarized pulsed RF field, applied at a resonant frequency, forces spin transitions between the two orientations. Energy is imparted into the nuclei during the spin transitions. The imparted energy is radiated from the nuclei as the nuclei “relax” to their previous magnetic state. The radiated energy is received by a receiving coil and processed to determine the characteristics of the tissue from which the radiated energy originated to generate the intra-body images.

Currently, most neurostimulation systems are designated as being contraindicated for MRI, because the time-varying magnetic RF field causes the induction of current which, in turn, can cause significant heating of patient tissue due to the presence of metal in various system components. The induced current can be “eddy current” and/or current caused by the “antenna effect.” As used herein, the phrase “MRI-induced current” refers to eddy current and/or current caused by the antenna effect.

“Eddy current” refers to current caused by the change in magnetic flux due to the time-varying RF magnetic field across an area bounding conductive material (i.e., patient tissue). The time-varying magnetic RF field induces current within the tissue of a patient that flows in closed-paths. When conventional pulse generator 103 (as shown in FIG. 1) and conventional implantable lead 104 are placed within tissue in which eddy currents are present, the implantable lead and the pulse generator provide a low impedance path for the flow of current. Electrodes 102 of the lead provide conductive surfaces that are adjacent to current paths 101 within the tissue of the patient. The electrodes 102 are coupled to the pulse generator 103 through a wire conductor within the implantable lead 104. The metallic housing (the “can”) of the pulse generator 103 provides a conductive surface in the tissue in which eddy currents are present. Thus, current can flow from the tissue through the electrodes 102 and out the metallic housing of the pulse generator 103. Because of the low impedance path and the relatively small surface area of each electrode 102, the current density in the patient tissue adjacent to the electrodes 102 can be relatively high. Accordingly, resistive heating of the tissue adjacent to the electrodes 102 can be high and can cause significant, irreversible tissue damage.

Also, the “antenna effect” can cause current to be induced which can result in undesired heating of tissue. Specifically, depending upon the length of the stimulation lead and its orientation relative to the time-varying magnetic RF field, the wire conductors of the stimulation lead can each function as an antenna and a resonant standing wave can be developed in each wire. A relatively large potential difference can result from the standing wave thereby causing relatively high current density and, hence, heating of tissue adjacent to the electrodes of the stimulation lead.

### SUMMARY

In one embodiment, a percutaneous stimulation lead for electrically stimulating tissue of a patient, comprises: a lead body of insulative material; a plurality of conductors within the lead body; a plurality of terminals for receiving electrical pulses disposed on a proximal portion of the lead body; and a plurality of electrodes disposed on a distal portion of the lead body, the plurality of electrodes being electrically coupled to the plurality of terminals through the plurality of conductors, wherein each electrode comprises a respective first surface exposed on an exterior surface of the stimulation lead to conduct current to or from tissue of the patient and a respective second surface disposed within an interior of the stimulation lead, the plurality of electrodes are arranged such that adjacent pairs of electrodes are capacitively coupled through a first surface of a first electrode of the respective pair and a respective second surface of a second electrode of the respective pair to substantially block current flow between adjacent electrodes at stimulation frequencies and to substantially pass current between adjacent electrodes at MRI frequencies.

The foregoing has outlined rather broadly certain features and/or technical advantages in order that the detailed description that follows may be better understood. Additional features and/or advantages will be described hereinafter which form the subject of the claims. It should be appreciated by those skilled in the art that the conception and specific embodiment disclosed may be readily utilized as a basis for modifying or designing other structures for carrying out the same purposes. It should also be realized by those skilled in the art that such equivalent constructions do not depart from the spirit and scope of the appended claims. The novel features, both as to organization and method of operation, together with further objects and advantages will be better understood from the following description when considered in connection with the accompanying figures. It is to be expressly understood, however, that each of the figures is provided for the purpose of illustration and description only and is not intended as a definition of the limits of the appended claims.

### BRIEF DESCRIPTION OF THE DRAWINGS

FIG. 1 depicts a pulse generator and implantable lead subjected to eddy current induced by the time-varying RF field of a MRI scan.

FIG. 2 depicts a portion of a stimulation lead adapted to mitigate MRI induced current according to one representative embodiment.

FIG. 3 depicts a circuit diagram associated with the electrode of the lead shown in FIG. 2 according to one representative embodiment.

FIG. 4 depicts another circuit diagram for mitigating MRI induced current according to one representative embodiment.

FIG. 5 depicts electrode assemblies connected to a lead body to implement the circuit shown in FIG. 4.

FIG. 6 depicts a circuit for mitigating MRI induced current according to an alternative embodiment.

FIG. 7 depicts a lead for implementing the MRI filtering circuitry shown in FIG. 6 according to one representative embodiment.

FIG. 8 depicts a distal end of a stimulation lead according to one representative embodiment.

FIG. 9 depicts a stimulation lead including the distal end shown in FIG. 8 according to one representative embodiment.

FIG. 10 depicts a stimulation system according to one representative embodiment.

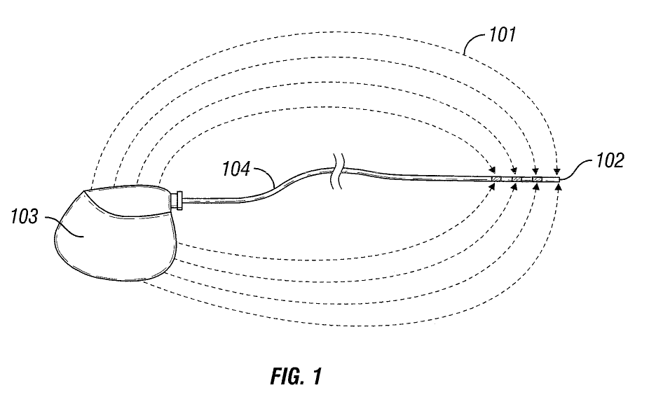

### DETAILED DESCRIPTION

Some representative embodiments are directed to a MRI compatible lead for stimulation of a patient. Specifically, some representative embodiments provide passive electrical components within the hollow volume defined by a “wrapped around” electrode of a percutaneous lead. Preferably, an inductor is provided within the space defined by the electrode. Additionally, a capacitive reactance also connects one end of the inductor to the electrode. The values of the inductance and capacitance of the passive electronic components are preferably selected based upon the expected operating frequency (f) of a particular class of MRI systems. By inserting a series tuned LC impedance between one electrode and the IPG, MRI induced current between the electrode and the IPG may be reduced. Although a tuned LC circuit is employed according to one representative embodiment, other embodiments may implement other MRI-induced current filtering circuits using passive electrical components within the confines of the volume defined by an electrode of the lead.

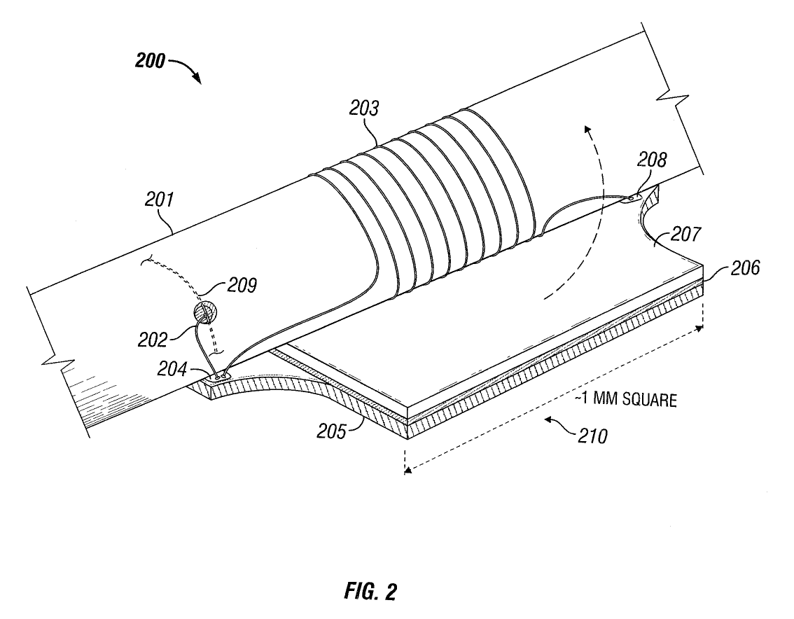

FIG. 2 depicts lead 200 that comprises passive electronic components within the confines of the space defined by a band or ring-like electrode for mitigating MRI induced current according to one representative embodiment. Lead 200 comprises lead body 201 of insulative material. The insulative material of lead body 201 encloses or encapsulates the wire conductors (including conductor 209) that conduct electrical pulses between the electrodes and terminals of the lead. Lead body 201 can be fabricated using any conventional or known fabrication technique or any later developed technique. An example of a suitable fabrication technique for forming a lead body 201 with embedded wire conductors can be found in U.S. Pat. No. 7,149,585 which is incorporated herein by reference.

Lead 200 comprises capacitive electrode assembly 210. Capacitive electrode assembly 210 comprises electrode 205, a layer of dielectric material 206, and interior metal component or layer 207. Electrode 205, dielectric material 206, and interior metal component 207 are shown in a flat configuration in FIG. 2 for the sake of clarity. After the completion of the fabrication of lead 201, electrode 205, dielectric material 206, and interior metal component 207 are preferably disposed in a band or ring-like manner around lead body 201.

Electrode 205 is disposed on the exterior of capacitive electrode assembly 210 to provide electrical stimulation from the IPG to tissue of the patient. Electrode 205 is preferably fabricated using platinum or a platinum-iridium alloy, although any suitably conductive and biostable, biocompatible material may be employed. Interior metal component 207 can be fabricated using a similar conductive material.

Dielectric material 206 electrically insulates electrode 205 from interior metal component 207. In one embodiment, the thickness of dielectric material is approximately 100 microns, although any suitable thickness may be employed. Suitable materials for dielectric material 206 include materials commonly utilized in lead fabrication technologies such as polyurethanes, silicone-based materials (e.g., PurSil™ and CarboSil™), polyethylene, polyimide, polyvinylchloride, PTFT, EFTE, etc.

In this embodiment, capacitive electrode assembly 210 provides the capactive reactance for an LC circuit as discussed above. The capacitance of the electrode 205, dielectric material 206, and interior component 207 is approximately equal to: C=εA/d, where ε is the permittivity of the dielectric material, A is the surface area of interior metal component 207, and d is the thickness of the dielectric material.

Thin wire 203 is wrapped around a region of lead body 201 to form an inductor. Upon completion of the fabrication of lead 200, wire 203 is preferably enclosed by interior metal layer 207, dielectric material 206, and electrode 205. Wire 203 is preferably coated with an insulative polymer or other suitable insulator. The insulative material at one end of wire 203 is stripped and the end of wire 203 is preferably welded to electrode at location 204. The insulative material at the other end of wire is also stripped and the other end is welded to interior metal component 207 at location 208. Wire 203 comprises a number of turns about lead body 201 between location 204 and location 208. The inductance provided by the inductor is related to the number of turns of the wire and the outside diameter of lead body 301. The inductance can be estimated by the following equation: L=μ₀μᵣN²A/l, where μ₀ is the permeability of free space, μᵣ is the permeability of the lead body, N is the number of turns of the wire, A is the cross sectional area of the lead, and L is the length of the portion of wire that is wrapped about the lead body.

Additionally, jumper wire 202 is welded to interior metal component 207 at location 208. Jumper wire 202 is used as a convenient intermediate electrical connector to connect to wire conductor 209 that is embedded within the lead body 301. Preferably, a small aperture is formed in the insulative material of lead body 301 using a suitable laser to expose a small portion conductor 209. One end of jumper wire 202 is placed within the aperture and welded to conductor 209 at that location. The other end of jumper wire 202 is then welded to interior metal component 207 at location 208. Jumper wire 202 is also preferably maintained underneath electrode assembly 210 upon completion of the fabrication of lead 200.

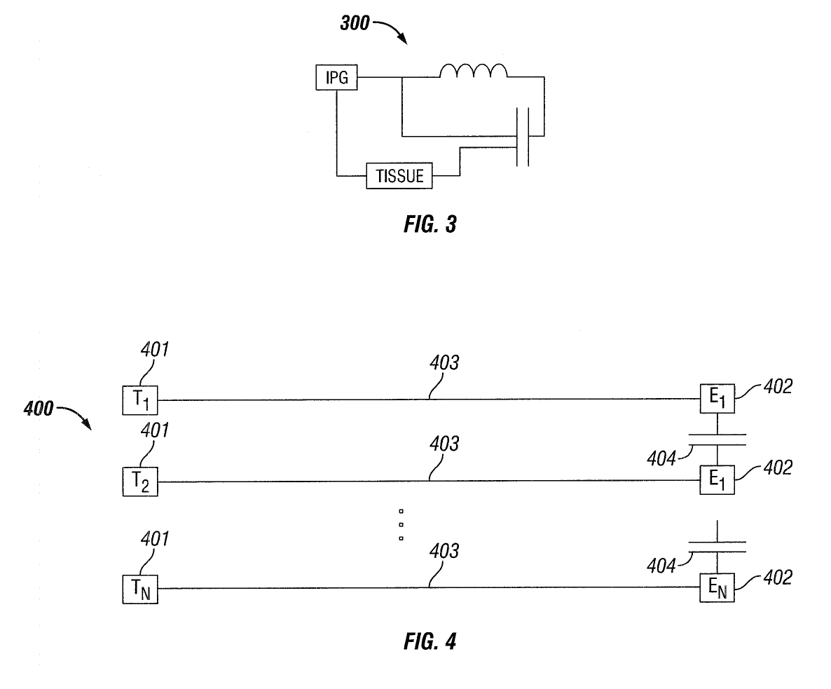

FIG. 3 depicts equivalent circuit representation 300 for the lead shown in FIG. 2. Circuit 300 includes a series LC component as formed by capacitive electrode assembly 210 and wire wrapped inductor 203. As seen in FIG. 3, one plate of the capacitor is coupled to the IPG and the tissue of the patient while the other plate of the capacitor is coupled to one end of the inductor. Additionally, a return path is shown from a plate of the capacitor through tissue of the patient to the IPG. Preferably, the capacitance and inductance of circuit 300 are selected to resonate at a particular frequency that corresponds to an anticipated MRI operating frequency (e.g., 63.9 MHz). The appropriate values for resonance at the MRI operating frequency can be estimated using the following equation: f=1/(2*n*sqrt(L×C)), where f is MRI operating frequency, L is the inductance provided by the wrapped wire, and C is the capacitance of electrode assembly 210.

Other circuit designs may be employed to reduce MRI induced current according to other representative embodiments. FIG. 4 depicts circuit diagram 400 for mitigating MRI induced current according to one representative embodiment. Circuit diagram 400 depicts a plurality of terminals 401 (T₁-T_{N}) electrically coupled to a plurality of electrodes 402 (E₁-E_{N}) through lead wires 403. Each electrode 402 is electrically coupled through a respective capacitor 404 to the next electrode 402 (e.g., electrode E₁ is electrically coupled through a capacitor to electrode E₂). The capacitance of the capacitors 404 is selected such that capacitors exhibit a relatively high impedance at stimulation frequencies (e.g., at or below 1000 Hz, 2000 Hz, or 3000 Hz) and a relatively low impendence at MRI frequencies (e.g., 63.9 MHz or above). When electrodes 402 are electrically coupled in this manner, a reduction in MRI induced heating has been observed.

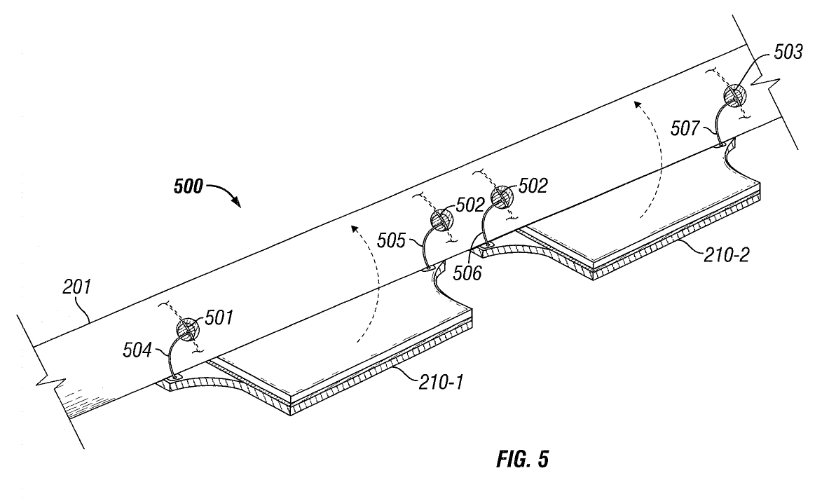

FIG. 5 depicts electrode assemblies 210 connected to lead body 201 to implement the circuit shown in FIG. 4. As is known in the art, wire conductors embedded with lead body 201 are typically helically wound and are accessible at many locations along lead body 201. Some representative embodiments utilize the helical arrangement of wire conductors to couple an electrode assembly 210 to multiple wire conductors to capacitively couple adjacent electrodes together. Specifically, as shown in FIG. 5, the electrode portion of electrode assembly 210 is coupled to wire conductor 501 of lead body 201 through jumper wire 504. Wire conductor 501 is also coupled to a terminal (not shown) at the proximal end of lead body 201. The interior metal component of electrode assembly 210-1 is coupled to wire conductor 502 through jumper wire 505. Wire conductor 502 is also coupled (at another location) through jumper wire 506 to the electrode of electrode assembly 210-2. Wire conductor 502 connects the electrode of electrode assembly 210-2 to another terminal (not shown) at the proximal end of lead body 201. In a similar manner, the interior metal component of electrode assembly 210-2 is coupled to wire conductor 503 (through jumper wire 507) that is used to connect the next electrode to a terminal.

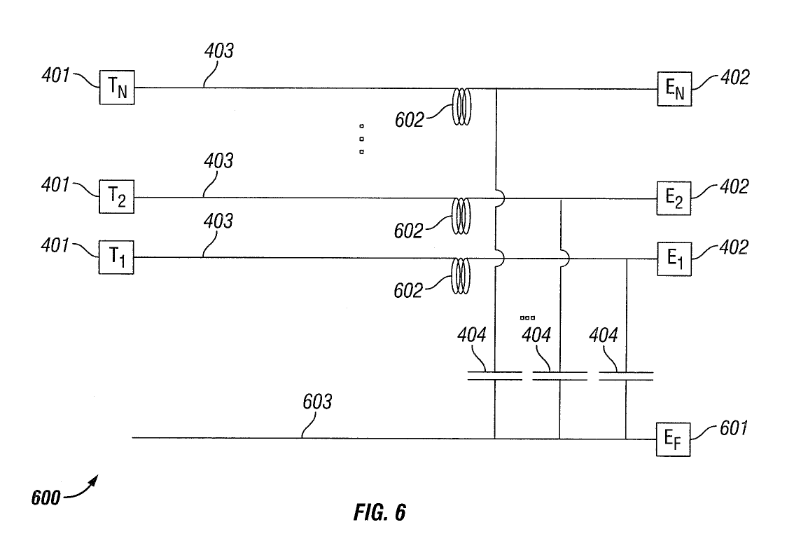

FIG. 6 depicts circuit 600 for mitigating MRI induced current according to an alternative embodiment. Circuit diagram 600 depicts a plurality of terminals 401 (T₁-T_{N}) electrically coupled to a plurality of electrodes 402 (E₁-E_{N}) through lead wires 403. Additionally, inductors 602 are disposed between lead wires 403 and electrodes 402 to limit the current flowing therebetween at high frequencies. Specifically, the inductance of inductors 602 is preferably selected such that relatively little attenuation occurs at stimulation frequencies while a relatively high amount of attenuation occurs at MRI frequencies. In addition, the electrodes 402 (E₁-E_{N}) are coupled through capacitors 404 to line 603 which leads to floating electrode 601 (E_{F}). The capacitance of capacitors 404 is preferably selected such that the impedance is relatively high at stimulation frequencies while the impedance is relatively low at MRI frequencies. Floating electrode 601 preferably provides a relatively large surface area relative to the other electrodes 402. At MRI frequencies, the MRI induced current will be distributed over a greater surface area and any accompanying temperature rise in patient tissue will be reduced.

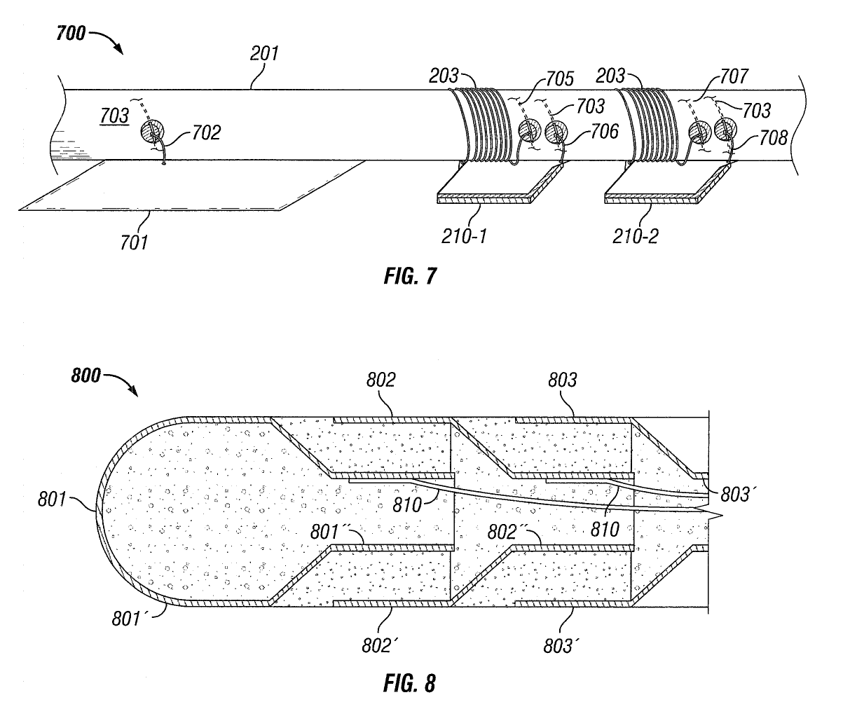

FIG. 7 depicts lead 700 for implementing the MRI filtering circuitry shown in FIG. 6 according to one representative embodiment. As shown in FIG. 7, lead 700 comprises floating electrode 701 that possesses a relatively large surface area. Floating electrode 701 is coupled to wire conductor 703 that is embedded within lead body 201 using jumper wire 702. Wire conductor 703 need not necessarily be coupled to a terminal on the proximal end of lead body 201. Lead 700 further comprises a plurality of electrodes assemblies 210 (shown as 210-1 and 210-2). Wire conductor 705, embedded within lead body 201, is coupled to one end of thin wire 203. The other end of thin wire 203 is coupled to the electrode portion of electrode 210-1. The interior metal component of electrode assembly 210-1 is coupled to wire conductor 703 of lead body 201 using jumper wire 706. Electrode assembly 210-2 is disposed in substantially the same manner as electrode assembly 210-1. The electrode portion of electrode assembly 210-2 is coupled to one end of thin wire 203. The other end of thin wire 203 is coupled to wire 707, which is embedded in lead body 201. Also, the interior metal component of electrode assembly 210-2 is coupled to wire 703 of lead body 201 using jumper wire 708.

FIG. 8 depicts an internal cross-sectional view of the very distal-end portion of stimulation lead 800 according to another representative embodiment. Stimulation lead 800 comprises a plurality of electrodes (only electrodes 801, 802, and 803 are shown in FIG. 8) which are disposed in succession at the distal end of the lead. Each electrode comprises a respective first surface (surfaces 801′, 802′ and 803′ are shown in FIG. 8) that is exposed on an exterior of the lead body. The first surface is utilized to conduct stimulation pulses from conductors 810 of the stimulation lead 800 to tissue of the patient. Also, each electrode comprises a respective second surface (surfaces 801″, 802″, and 803″ are shown in FIG. 8) that is disposed within the insulative material of the lead body.

The electrodes are arranged in such a manner that the respective surfaces of the electrodes are capacitively coupled. For example, as shown in FIG. 8, surface 801″ and surface 802′ are capactively coupled and surface 802″ and surface 803′ are capactively coupled. The capacitance between the respective surfaces is defined by the surface area of the respective surfaces, the distance between the surfaces, and the dielectric constant of the insulative material of the lead body. Preferably, these characteristics are adapted to substantially block current flow between the surfaces at stimulation frequencies and to substantially permit current flow between the surfaces at MRI frequencies. In one preferred embodiment, the capacitance between each pair of electrodes is at least 3.8 pF.

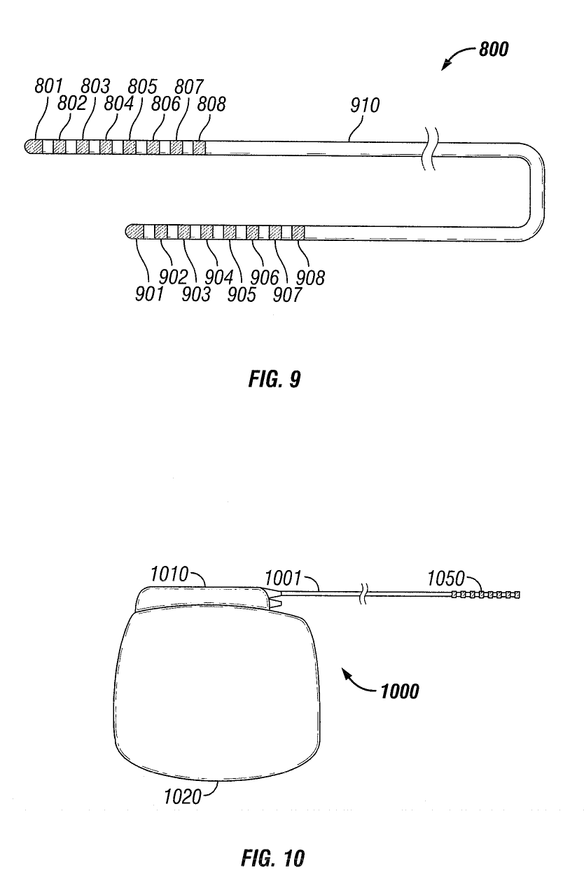

FIG. 9 depicts another view of stimulation lead 800. As shown in FIG. 9, stimulation lead 800 comprises eight electrodes (shown as electrodes 801-808) in total. Each adjacent pair of electrodes are capacitively coupled in the manner shown in FIG. 8. Electrodes 801-808 are electrically coupled to terminals 901-908 through the conductors (not shown) embedded within the lead body of stimulation lead 800. Although eight electrodes 801-808 and terminals 901-908 are shown, any suitable number of electrodes and terminals can be provided according to other embodiments. As shown in FIG. 9, stimulation lead 800 further comprises conductive sheath 910. Conductive sheath 910 is adapted to contact tissue of the patient. In one representative embodiment, conductive sheath 910 is implemented using a conductive polymer (e.g., a Carbosil® material with PtIr particles embedded therein) applied to the exterior of the lead body. Conductive sheath 910 may also be implemented using a relatively flexible biocompatible metal sheath. Alternatively, one or more windings of small diameter wires could be utilized to implement conductive sheath 910.

The conductive sheath 910 is preferably electrically coupled to electrode 808 via a capacitance. At stimulation frequencies, electrodes 801-808 and conductive sheath 910 are preferably isolated from each other by a relatively high impedance. At MRI frequencies, electrodes 801-808 and conductive sheath 910 are preferably electrically coupled by a relatively low impedance. Thus, MRI induced current is substantially distributed across the surface defined by electrodes 801-808 and conductive sheath 910. Because of the larger surface area formed by electrodes 801-808 and conductive sheath 910, the current density associated with MRI induced current is lowered and, hence, MRI heating is reduced. As shown in FIG. 9, conductive sheath 910 preferably extends along a substantial length of the lead body of stimulation lead 800. Conductive sheath 910 could alternatively occupy a lesser amount of the length of the lead body so long as conductive sheath 910 possesses sufficient surface area to reduce or mitigate MRI heating.

In one alternative embodiment, conductive sheath 910 may be similarly capacitively coupled to a most distal terminal 908 of the plurality of terminals 901-908. Also, in this alternative embodiment, each adjacent pair of terminals are capacitively coupled in the same manner as adjacent electrodes of lead 800.

FIG. 10 depicts stimulation system 1000 according to one representative embodiment. Neurostimulation system 1000 includes pulse generator 1020 and one or more stimulation leads 1001. An example of a commercially available pulse generator is the EON® pulse generator available from Advanced Neuromodulation Systems, Inc. Pulse generator 1020 is typically implemented using a metallic housing that encloses circuitry for generating the electrical pulses for application to neural tissue of the patient Control circuitry, communication circuitry, and a rechargeable battery (not shown) are also typically included within pulse generator 1020. Pulse generator 1020 is usually implanted within a subcutaneous pocket created under the skin by a physician.

Lead 1001 is electrically coupled to the circuitry within pulse generator 1020 using header 1010. Lead 1001 is used to conduct the electrical pulses from the implant site of the pulse generator for application to the targeted nerve tissue. For example, the distal end of lead 1001 may be positioned within the epidural space of the patient to deliver electrical stimulation to spinal nerves to treat chronic pain of the patient. Also, an “extension” lead (not shown) may be utilized as an intermediate connector if deemed appropriate by the physician. Electrodes 1050 are preferably coupled to the conductor wires of lead 1001 in a manner that reduces MRI induced current or otherwise mitigates MRI heating using one or more of the techniques discussed above. Also, inductive wires (not shown) may be employed underneath electrodes 1050 to reduce MRI induced current or otherwise mitigate MRI heating.

Some representative embodiments may provide a number of advantages. Some representative embodiments provide an efficient fabrication methodology for inclusion of MRI current mitigating components within a stimulation lead. For example, some representative embodiments do not complicate the lead body of stimulation lead to accommodate passive MRI mitigating components as seen in some proposed MRI compatible lead designs. Additionally, some representative embodiments provide partial shielding for the magnetic core of the inductor thereby reducing distortion within MRI imaging caused by the stimulation lead.

Although some embodiments have been described in terms of neurostimulation systems, the present application is not limited to such systems. For example, leads for cardiac applications (e.g., pacing, defibrillation, etc.) could be adapted to mitigate MRI induced current for alternative embodiments.

Although certain representative embodiments and advantages have been described in detail, it should be understood that various changes, substitutions and alterations can be made herein without departing from the spirit and scope of the appended claims. Moreover, the scope of the present application is not intended to be limited to the particular embodiments of the process, machine, manufacture, composition of matter, means, methods and steps described in the specification. As one of ordinary skill in the art will readily appreciate when reading the present application, other processes, machines, manufacture, compositions of matter, means, methods, or steps, presently existing or later to be developed that perform substantially the same function or achieve substantially the same result as the described embodiments may be utilized. Accordingly, the appended claims are intended to include within their scope such processes, machines, manufacture, compositions of matter, means, methods, or steps.

1. A percutaneous stimulation lead for electrically stimulating tissue of a patient, comprising:
a lead body of insulative material;
a plurality of conductors within the lead body;
a plurality of terminals for receiving electrical pulses disposed on a proximal portion of the lead body; and
a plurality of electrodes disposed on a distal portion of the lead body, the plurality of electrodes being electrically coupled to the plurality of terminals through the plurality of conductors, wherein each electrode comprises a respective first surface exposed on an exterior surface of the stimulation lead to conduct current to or from tissue of the patient and a respective second surface disposed within an interior of the stimulation lead, the plurality of electrodes are arranged such that adjacent pairs of electrodes are capacitively coupled through a first surface of a first electrode of the respective pair and a respective second surface of a second electrode of the respective pair to substantially block current flow between adjacent electrodes at stimulation frequencies and to substantially pass current between adjacent electrodes at MRI frequencies.

2. The percutaneous stimulation lead of claim 1 further comprising:
a conductive sheath disposed on a medial portion of the lead body, wherein one electrode of the plurality of electrodes is capacitively coupled to the conductive sheath to substantially block current flow at stimulation frequencies and to substantially pass current at MRI frequencies.

3. The percutaneous stimulation lead of claim 2 wherein a most proximal electrode of the plurality of electrodes is capacitively coupled to the conductive sheath.

4. The percutaneous stimulation lead of claim 2 wherein the conductive sheath extends over a majority of a length of the lead body.

5. The percutaneous stimulation lead of claim 2 wherein the conductive sheath is formed of a conductive flexible polymer material applied to an exterior of the lead body.

6. The percutaneous stimulation lead of claim 2 wherein the conductive sheath is capacitively coupled to the plurality of terminals, the capacitance between the conductive sheath and the plurality of terminals substantially blocks current flow at stimulation frequencies and substantially passes current at MRI frequencies.

7. The percutaneous stimulation lead of claim 1 wherein a capacitance between each adjacent pair of electrodes is at least 3.8 pF.

8. The percutaneous lead of claim 1 wherein the plurality of electrodes form an inner channel extending along a distal portion of the stimulation lead, wherein the plurality of conductors are disposed within the inner channel at the distal portion of the stimulation lead.

9. The percutaneous lead of claim 1 wherein insulative material of the lead body is disposed between the respective first and second surfaces of each pair of adjacent electrodes of the plurality of electrodes.

10. The percutaneous lead of claim 1 wherein the respective first and second surfaces are substantially annular surfaces.
